(19) United States
(12) Patent Application Publication
(10) Pub. No.: US 2025/0387111 A1
(43) Pub. Date: Dec. 25, 2025

(54) TISSUE RETRACTOR AND ADAPTOR THEREFOR

(71) Applicant: CoreLink, LLC, St. Louis, MO (US)

(72) Inventors: Tim Stiefferman, House Springs, MO (US); Brett Smith, St. Louis, MO (US); Adam MacMillan, Quincy, MA (US)

(21) Appl. No.: 19/256,052

(22) Filed: Jun. 30, 2025

Related U.S. Application Data
(63) Continuation of application No. 17/806,651, filed on Jun. 13, 2022, now Pat. No. 12,343,001.
(60) Provisional application No. 63/209,713, filed on Jun. 11, 2021.

(51) Int. Cl. A61B 17/02; A61B 17/00

(52) U.S. Cl. CPC A61B 17/0206; A61B 2017/00407; A61B 2017/00473

(57) ABSTRACT

A retractor for spine surgery includes a body. Left, right, and center retracting arms are coupled to the body. Left, right, and center blades are operatively coupled to proximal ends of the respective retracting arms. A retractor adaptor is configured to couple the retractor to a mount.

### CROSS-REFERENCE TO RELATED APPLICATIONS

The present application is a continuation application of U.S. patent application Ser. No. 17/806,651, filed Jun. 13, 2022, which claims priority to U.S. Provisional Ser. No. 63/209,713, filed Jun. 11, 2021, the entirety of which is hereby incorporated by reference.

### FIELD OF THE DISCLOSURE

The present disclosure generally relates to a tissue retractor and an adaptor for attaching the tissue retractor to a retractor holder.

### BACKGROUND OF THE DISCLOSURE

Tissue retractors are used in surgical procedures to provide an opening or access to a surgical site, such as a spine or other surgical site. The opening created by the tissue retractor enables a surgeon to insert surgical instruments and implants, for example, into the patient's body.

### SUMMARY

In one aspect, a retractor for spine surgery generally comprises: a body; left, right, and center retracting arms coupled to the body; and left, right, and center blades operatively coupled to proximal ends of the respective retracting arms.

In another aspect, a retractor adaptor configured to couple to a tissue retractor for spine surgery generally comprises: an adaptor body having proximal and distal ends, the proximal end of the adaptor body configured to couple to an arm mount; an annular row of teeth at the distal end of the adaptor body configured to mesh with an annular row of teeth of an adaptor mount of the tissue retractor; and a latch configured to releasably latch the retractor adaptor on the adaptor mount.

### BRIEF DESCRIPTION OF THE DRAWINGS

FIG. 1 is a perspective of a retractor shown in a closed, non-toed configuration.

FIG. 2 is a rear perspective of the retractor of FIG. 1.

FIG. 3 top plan view of the retractor of FIG. 1.

FIG. 4 is a perspective of the retractor shown in an open, non-toed configuration.

FIG. 5 is a top plan view of the retractor of FIG. 4.

FIG. 6 is an exploded view of a left arm and an associated pinion of the retractor.

FIG. 7 is a cross-sectional perspective of a right arm and an associated pinion of the retractor.

FIG. 8 is a top plan view similar to FIG. 3, but with a body of the retractor being removed from the retractor.

FIG. 9 is a cross-sectional perspective of the retractor showing cross sections of ratchet mechanisms.

FIG. 10 is an enlarged detail of FIG. 9.

FIG. 11 is similar to FIG. 10 showing the ratchet mechanism in a released configuration.

FIG. 12 is a cross-sectional perspective of the retractor in the closed, non-toed position.

FIG. 13 is similar to FIG. 12 with a center arm of the retractor in the open, non-toed position.

FIG. 14 is an enlarged, detail view of FIG. 13.

FIG. 15 is a side elevation of the retractor.

FIG. 16 a perspective of the retractor with a right blade exploded therefrom.

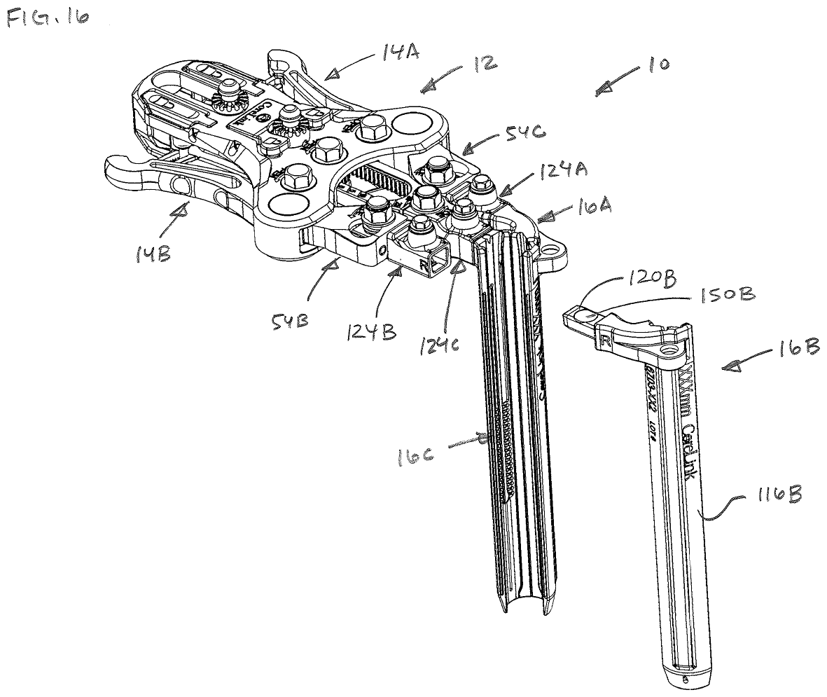

FIG. 17 is a cross-sectional perspective of the retractor with a distal end portion of the right arm in cross section.

FIG. 18 is an enlarged, detail view of FIG. 17.

FIG. 19 is a cross-sectional perspective of the retractor with sections taken through lug portions of the arms.

FIG. 20 is an enlarged, detail view of FIG. 19.

FIG. 21 is a perspective of a right blade of the retractor.

FIG. 22 is a cross section taken through line 22-22 in FIG. 15.

FIG. 23 is a perspective of an arm mount for a retractor.

FIG. 24 is a perspective of an actuator adaptor of the arm mount.

FIG. 25 is a perspective of the retractor and the actuator adaptor coupled thereto.

FIG. 26 is an enlarged, fragmentary cross section taken through the actuator adaptor and retractor of FIG. 25.

FIG. 27 is similar to FIG. 26, but with the actuator adaptor unlatched.

Corresponding reference characters indicate corresponding parts throughout the drawings.

### DETAILED DESCRIPTION OF THE DISCLOSURE

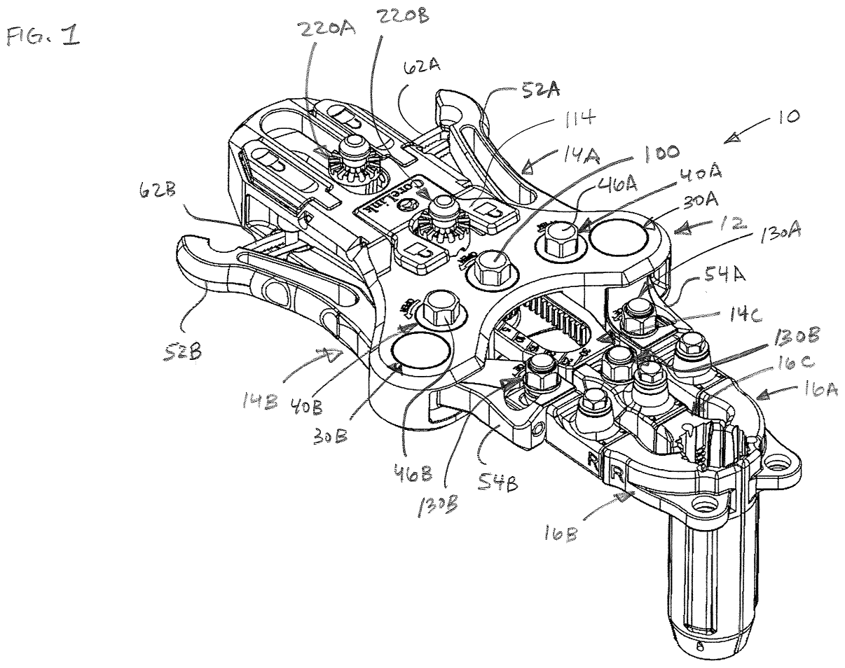

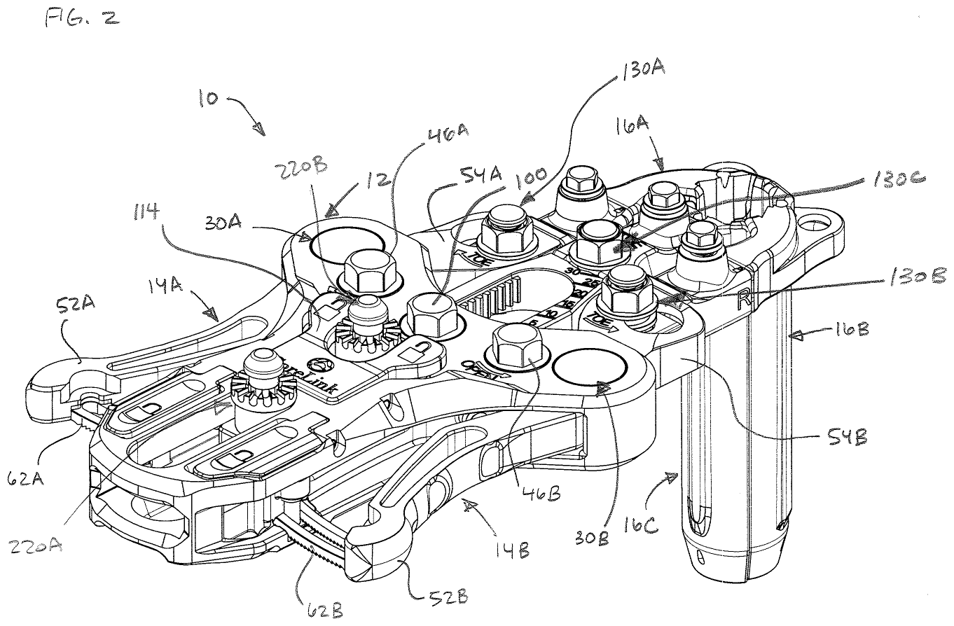

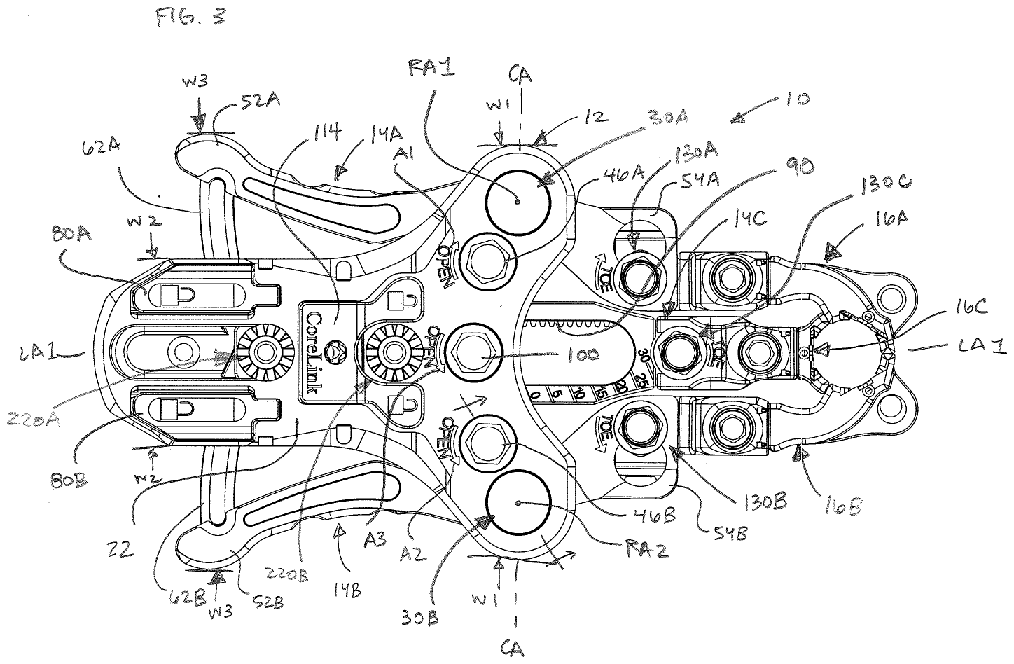

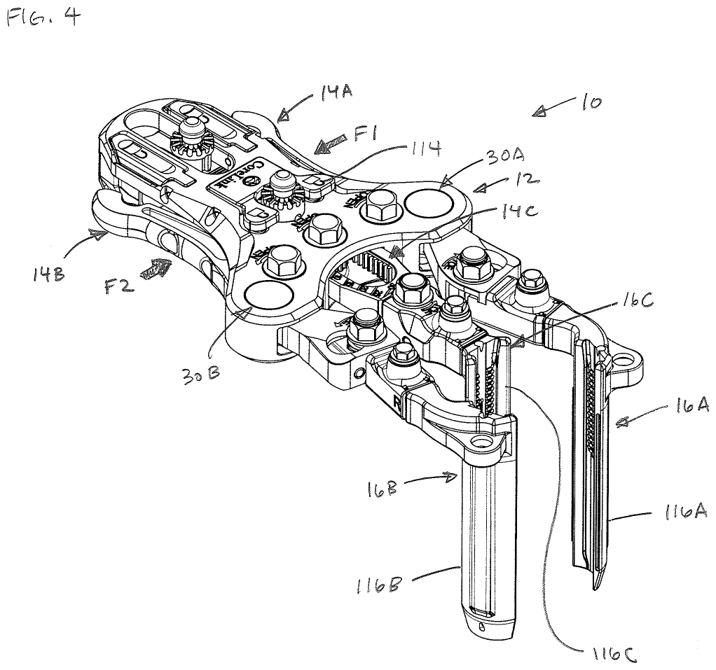

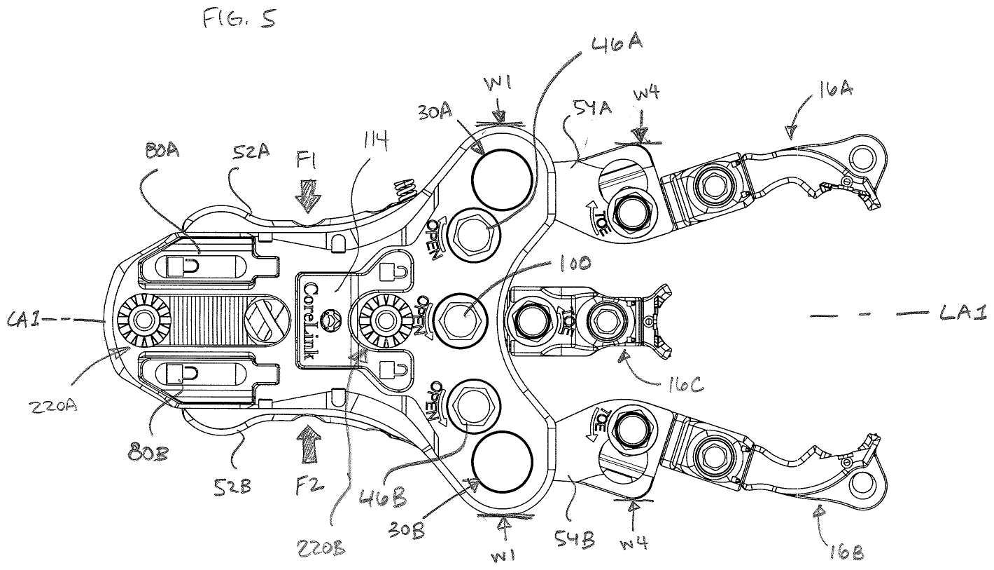

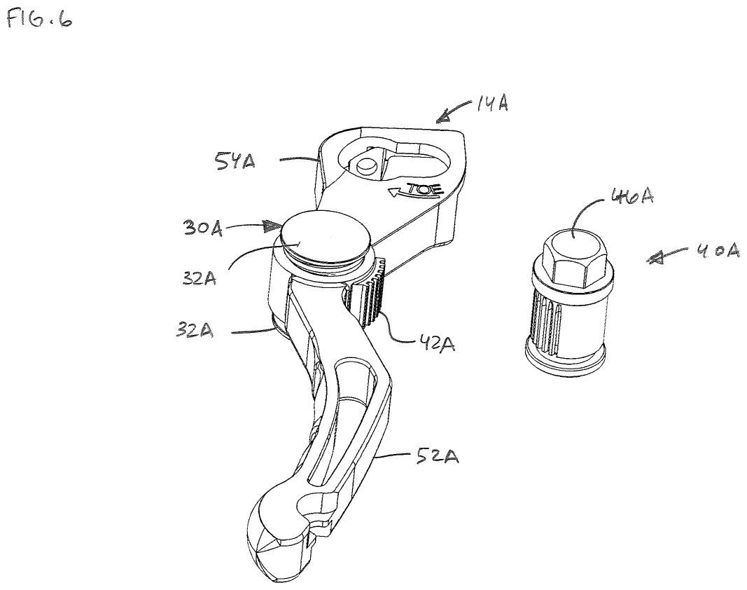

Referring now to the drawings, an illustrated embodiment of a tissue retractor constructed according to the teachings of the present disclosure is generally indicated at reference numeral 10. The tissue retractor 10 is operable to retract tissue during a surgical procedure, such as spinal surgery including but not limited to direct lateral access spine surgery (known as DLIF). The tissue retractor 10 has proximal and distal ends and a longitudinal axis LA1 (FIG. 3) extending between the proximal and distal ends. In general, the tissue retractor 10 includes a body, generally indicated at 12; left, right, and center retracting arms, generally indicated at 14A, 14B, and 14C, respectively, coupled to the body; and left, right, and center blades, generally indicated at 16A, 16B, and 16C, respectively, operatively coupled to proximal ends of the corresponding arms 14A, 14B, and 14C. In FIGS. 1-3, for example, the tissue retractor 10 is shown in its closed, non-toed configuration. In this configuration, the blades 16A, 16B, 16C are generally brought together circumferentially to form an elongate tube to enable insertion of the blades into an opening formed in the tissue to be retraced. In FIGS. 4 and 5, the tissue retractor 10 is shown in its open, non-toed configuration. In this configuration, the blades 16A, 16B, 16C are moved away from one another to thereby enable retraction of tissue. In FIG. 6, the tissue retractor 10 is shown in its expanded, toed configuration. In this configuration, the blades 16A, 16B, 16C are toed or canted to move distal ends of the blades further away from one another to thereby enable further retraction of tissue. The tissue-retractor 10 is operable between the different configurations during the surgical procedures, as explained in more detail below.

As shown best in FIG. 3, the body 20 is generally T-shaped or Y-shaped having a longitudinal portion 22 extending along the longitudinal axis LA1, and crosswise portion 24 extending crosswise of the longitudinal portion generally along a crosswise axis CA of the body. The crosswise portion 24, which is at a distal end of the longitudinal portion 22, has a width W1 greater than the width W2 of the longitudinal portion so that in general the width of the body 12 tapers proximally. As explained in more detail below, actuators, locks, and couplers are coupled to the body 12.

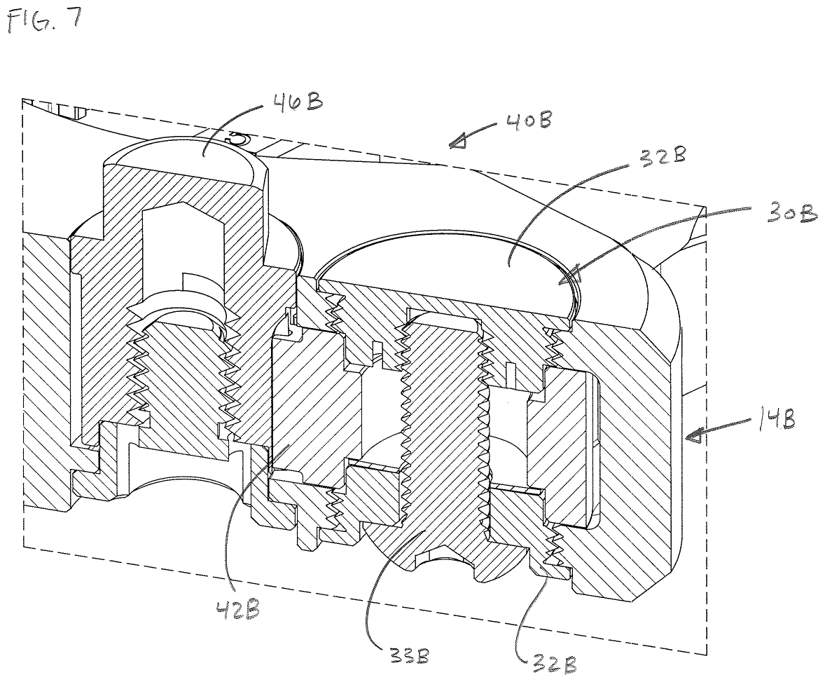

Referring to FIGS. 4 and 5, each of the retracting arms 14A, 14B, 14C are independently operable to selectively move the arms relative to the body 12. The left and right retracting arms 14A, 14B are selectively rotatable in a horizontal plane about respective axes RA1, RA2 relative to the body 12, thereby allowing the left and right blades 16A, 16B to be movable away from and toward one another in a generally lateral direction relative to the longitudinal axis LA1 between fully closed and fully opened positions. Each of the left and right retracting arms 14A, 14B is rotatably coupled to the body 12 by left and right pins, generally indicated at 30A, 30B, respectively. The left pin 30A is coupled to a left side portion of the crosswise portion 24, and the right pin 30B is coupled to the right side portion of the crosswise portion. In the illustrated embodiment, each pin 30A, 30B includes upper and lower bearings 32A, 32B and a fastener 33A, 33B (e.g., a screw or bolt) fastening the bearings to one another. As shown in FIG. 7, the upper bearing 32A, 32B interfaces with an upper portion of the body 12 and the lower bearing interfaces with a lower portion of the body, thereby enabling rotation of the corresponding retracting arm 14A, 14B about the respective axes RA1, RA2. As explained in more detail below, each of the left and right retracting arms 14A, 14B is selectively rotatable about the corresponding pin 30A, 30B through two different mechanisms: a rack and pinion mechanism and a lever mechanism.

As seen best in FIGS. 6 and 7, each rack and pinion mechanisms includes a pinion, generally indicated at 40A, 40B, respectively, rotatably coupled to the body 12, and a rack (e.g., an arcuate rack) 42A, 42B, respectively, fixed to or apart of the corresponding retracting arm 14A, 14B. The pinions 40A, 40B include teeth meshing with teeth of the respective racks 42A, 42B. Each of the pinions 40A, 40B includes a tool coupler 46A configured to couple to a tool (not shown) for driving rotation of the pinion. The illustrated tool coupler 46A, 46B comprises a hexagonal head configured to receive a hexagonal socket of a wrench or other tool for driving rotation. In the illustrated embodiment, rotation of the left pinion 40A in a clockwise direction (as indicated by arrow A1) opens the left blade 16A by rotating the left retracting arm 14A to move the left blade generally in a left direction away from the longitudinal axis LA1, and rotation of the right pinion 40B in a counterclockwise direction (as indicated by arrow A2) opens the right blade 16b by rotating the right retracting arm 14B to move the right blade generally in a right direction away from the longitudinal axis. Each of the pinions 40A, 40B are independently operable to independently open and close the left and right blade 16A, 16B.

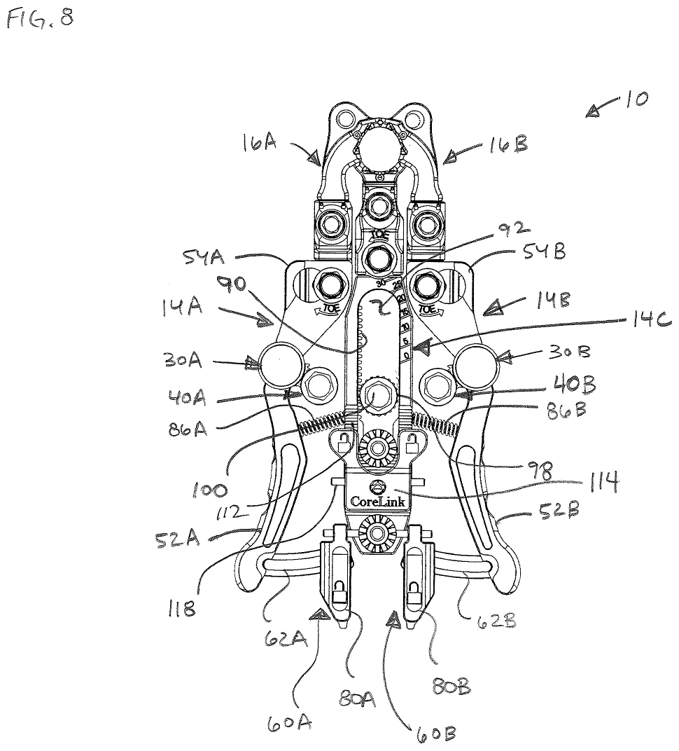

Referring to FIGS. 6 and 8, each lever mechanism includes an effort arm portion 52A, 52B, and a load arm portion 54A, 54B of the corresponding arm 14A, 14B. The effort arm portion 52A, 52B extends proximal of the corresponding pin 30A, 30B, and the load arm portion 54A, 54B extends distal of the corresponding pin. In use, as shown in FIGS. 4 and 5, generally lateral forces F1, F2 can be applied to one or both of the respective effort arm portions 54A, 54B of the left and right arms 14A, 14B to move the arm toward the longitudinal axis LA1. Movement of the effort arm portion 52A, 52B toward the longitudinal axis LA1 imparts movement of the corresponding load arm portion 54A, 54B away from the longitudinal axis. Thus, in one example the left and right effort arm portions 52A, 52B can be squeezed toward the longitudinal axis LA1 by a user's hand to manually open by hand the left and right blades 16A, 16B. Moving the left and right effort arm portions 52A, 52B toward the longitudinal axis LA reduces the crosswise footprint (e.g., width) extending crosswise between the left and right effort arm portions, while increasing the crosswise footprint (e.g., width) extending crosswise between the left and right load arm portions 54A, 54B. The left and right effort arm portions 52A, 52B may be configured to nest at least partially within the corresponding sides of the body 12 to further reduce the crosswise footprint therebetween. In the closed configuration (FIG. 3), the left and right effort arm portions 52A, 52B have a maximum crosswise footprint W3. This maximum crosswise footprint W3 may be less than or equal to the width W1 of the crosswise portion 24 of the body 12. In the open configuration (FIG. 5), the effort arm portions 54A, 54B have a maximum crosswise footprint W4. This maximum crosswise footprint W4 may be less than or equal to the width W1 of the crosswise portion 24 of the body 12.

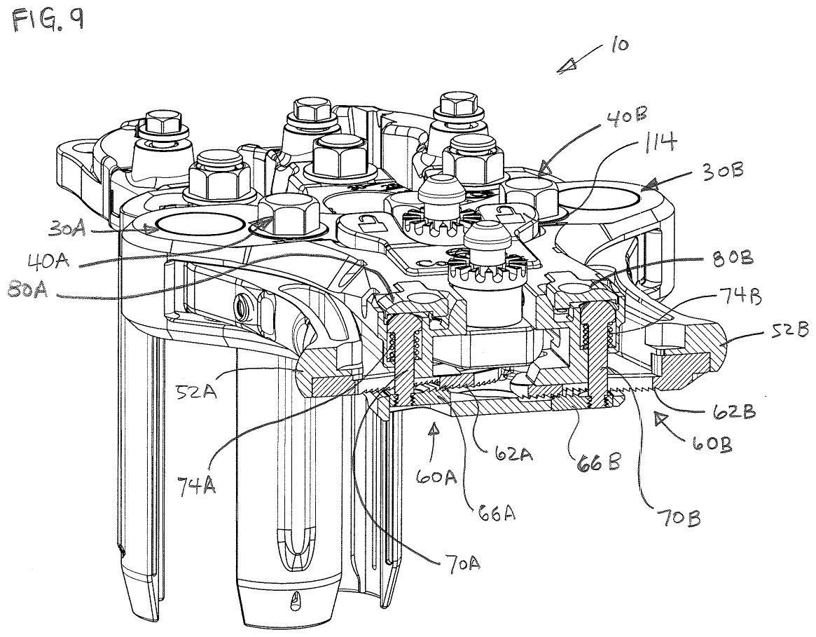

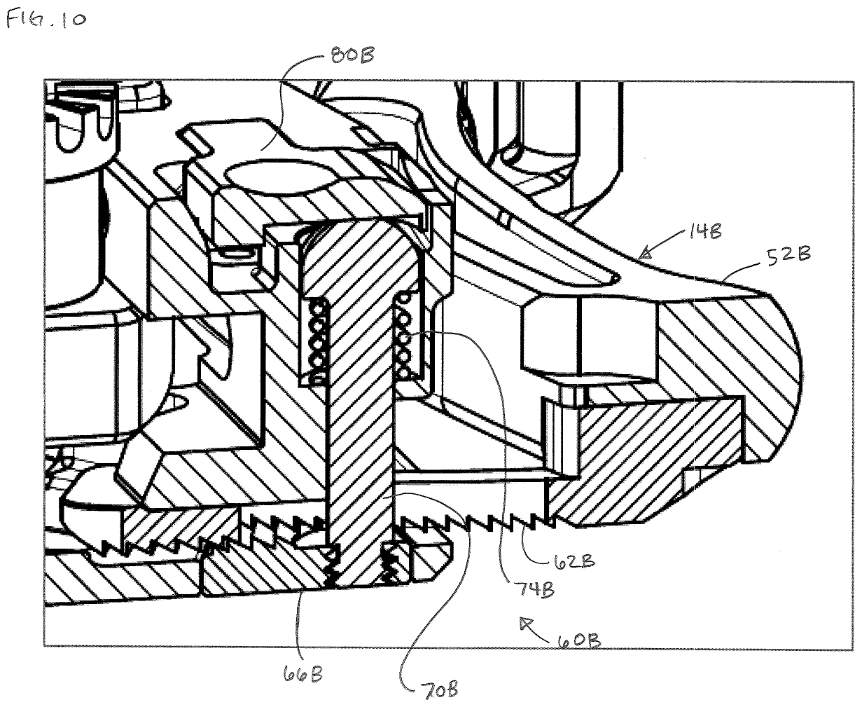

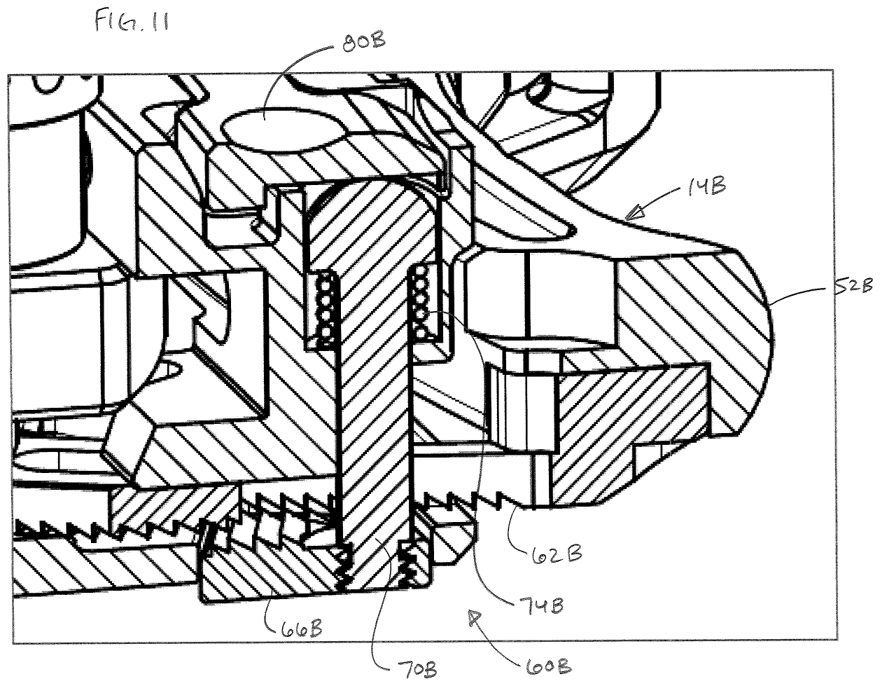

Each of the left and right retracting arms 14A, 14b has an anti-backoff mechanism associated therewith for independently inhibiting the left and right retracting arms from backing off its selective rotational position relative to the body 12. Referring to FIGS. 9-11, each anti-back off mechanism includes a ratchet mechanism, generally indicated at 60A, 60B, respectively. Each ratchet mechanism 60A, 60B respectively includes a ratchet rack 62A, 62B (e.g., a toothed rack) coupled to the corresponding one of the left and right retracting arms 14A, 14B and extending toward the longitudinal axis LA1, and a pawl 66A, 66B configured to engage (e.g., mesh) with the ratchet rack as the effort arm portion 52A, 52B is moved inward toward the longitudinal axis. Each pawl 66A, 66B is biased into meshing engagement with the ratchet rack 62A, 62B. A post 70A, 70B couples the pawl 66A, 66B to the body 12, and a spring 74A, 74B applying a biasing force on the post to bias the pawl toward the ratchet rack into meshing engagement therewith. Each ratchet mechanism 60A, 60B is configured to inhibit movement of the corresponding effort arm portions 52A, 52B laterally away from the longitudinal axis LA. Each ratchet mechanism 60A, 60B is further configured to be selectively released by disengaging the pawl 66A, 66B from the ratchet rack 62A, 62B. In the illustrated embodiment, each ratchet mechanism 60A, 60B includes a ratchet actuator 80A, 80B (e.g., a rotatable paddle or button) operable to move the post 70A, 70B against the biasing force of the spring 74A, 74B and move the pawl 66A, 66B away from the ratchet rack 62A, 62B. Referring to FIG. 8, closing springs 86A, 86B (e.g., compression springs) bias the respective left and right retracting arms 14A, 14B in the closed positions, such that the corresponding arm moves to the closed position under the force of the corresponding spring upon selective release of the pawl 66A, 66B from the ratchet rack 62A, 62B.

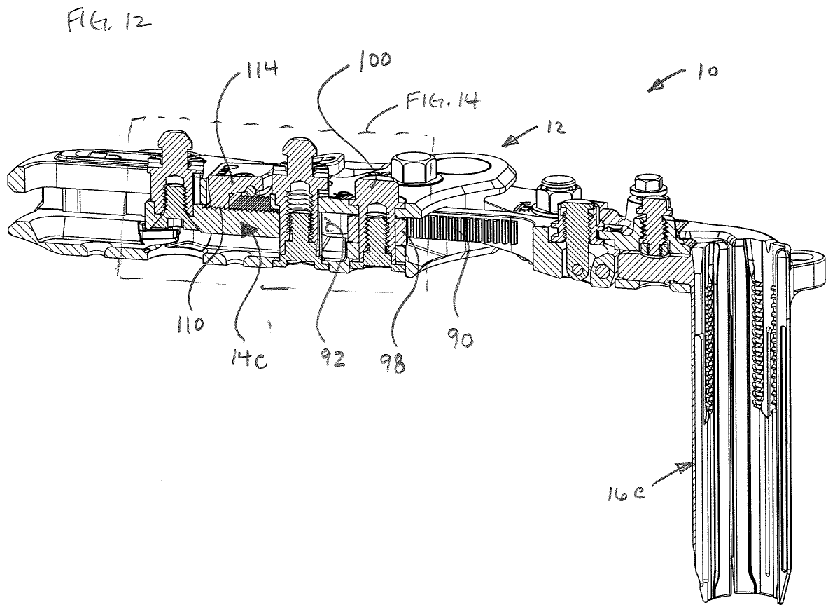

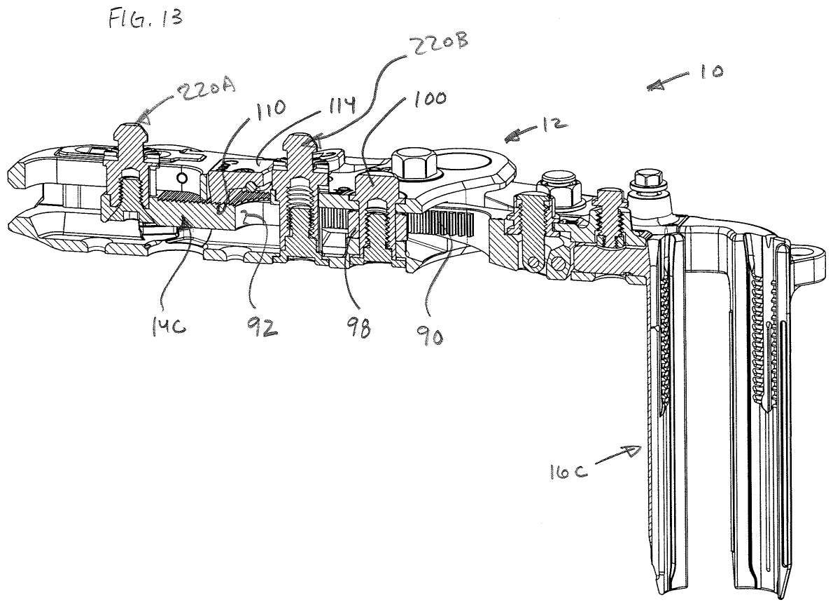

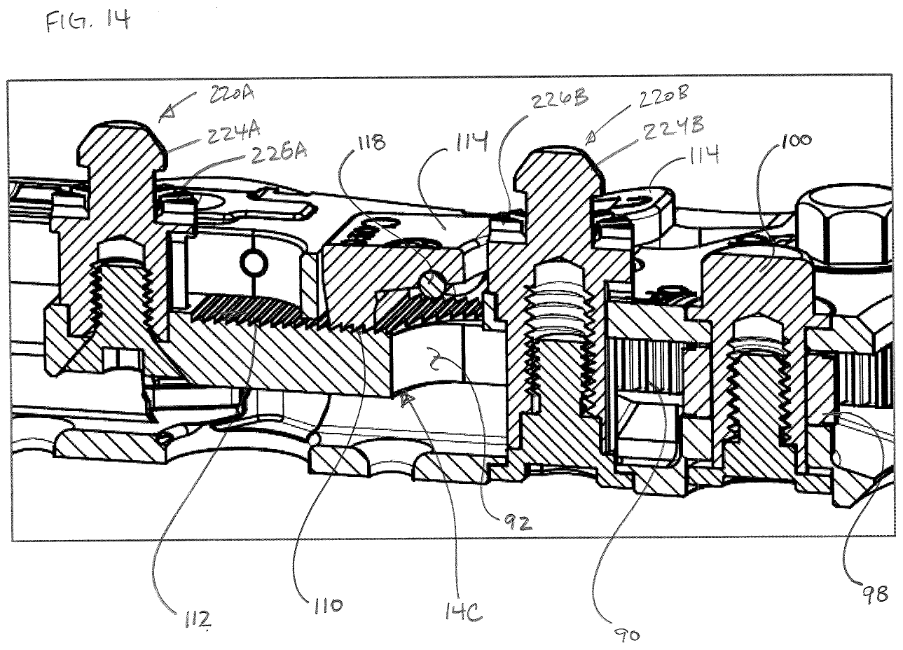

Referring to FIGS. 12-14, the center arm 14C is movable linearly generally along the longitudinal axis LA between fully a closed position (FIGS. 12 and 13) and a fully opened position (FIG. 14). In the illustrated embodiment, a rack and pinion mechanism enables movement of the center arm 14C relative to the body 12. The rack and pinion mechanism includes a toothed, linear rack 90 on the center retracting arm 14C, such as within a slot 92 defined by the center arm, and a pinion 98 rotatably coupled to the body 12 and meshing with the linear rack. The pinion 98 includes a pinion actuator or tool coupler 100 configured to couple to a tool (not shown) for driving rotation of the pinion. The illustrated tool coupler 100 comprises a hexagonal head configured to receive a hexagonal socket of a wrench or other tool for driving rotation. In the illustrated embodiment, rotation of the pinion 98 in a counterclockwise direction (as indicated by arrow A3 in FIG. 3) moves the center retracting arm 14C and the center blade 16C generally in a proximal direction.

Referring still to FIGS. 12-14, an anti-backoff mechanism inhibits unintentional movement of the center retracting arm 14C in a distal direction, thereby inhibiting incidental movement of the center retracting arm toward its closed position. The illustrated anti-backoff mechanism includes a detent 110 (e.g., a tooth member) configured to mesh with upper teeth 112 (e.g., an upper rack) on an upper surface of the center retracting arm. The detent 110 is biased to mesh with the upper teeth 112 by a spring (not shown) or other biasing element. A detent actuator 114 is coupled to the detent 110 and is hingedly coupled to the body 12 by one or more pins 118. The detent actuator 114 may comprise a paddle or button that rotates about the one or more pins 118 to selectively release the detent 110 from the upper teeth 112.

As can be seen from the drawings, each of the unlock or release actuators 80A, 80B, 114 (e.g., paddles or buttons) are accessible by the user at an upper surface of the body 12. Thus, the user may selectively release or unlock anyone of the arms 16A, 16B, 16C by having access to the upper surface of the body 12, and not requiring access to any sides or the bottom of the body. Moreover, the pinion actuators 46A, 46B, 100 for each of the pinions of the rack and pinion mechanisms are accessible at the upper surface of the body 12. In addition, the construction of the body 12 and the left and right arms 14A, 14B gives the retractor 10 a low profile and provides sufficient leverage when squeezing the arms to move the left and right blades 16A, 16B to the open positions.

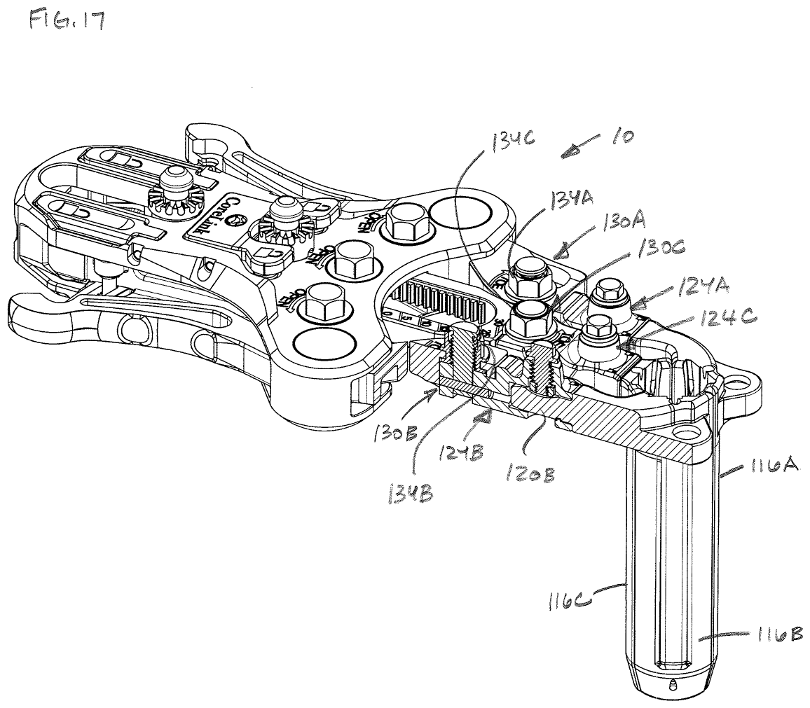

Referring to FIGS. 17-20, each of the blades 16A, 16B, 16C includes an arcuate blade body 116A, 116B, 116C, and a tongue 120A, 120B, 120C (i.e., a male coupling) extending proximally from the blade body. The tongue 120A, 120B, 120C is removably couplable to a lug connector, generally indicated at 124A, 124B, 124C, which in turn is coupled to the corresponding one of the retracting arms 14A, 14B, 14C. In the illustrated embodiment, each lug connector 124A, 124B, 124C, is coupled to the corresponding arm by a corresponding toeing mechanism, generally indicated at 130A, 130B, 130C, which is generally known in the art. Thus, the toeing mechanism 130A, 130B, 130C allows canting of the lug connector 124A, 124B, 124C, which in turn, toes the corresponding blade body 116A, 116B, 116C. The construction of the toeing mechanisms 130A, 130B, 130C may be similar to those known in the art, including an actuator 134A, 134B, 134C with a hexagonal head for coupling with a tool. The blade body 116A, 116B, 116C may have a selected longitudinal length and may have a selected cross-sectional arcuate length, as is generally known in the art.

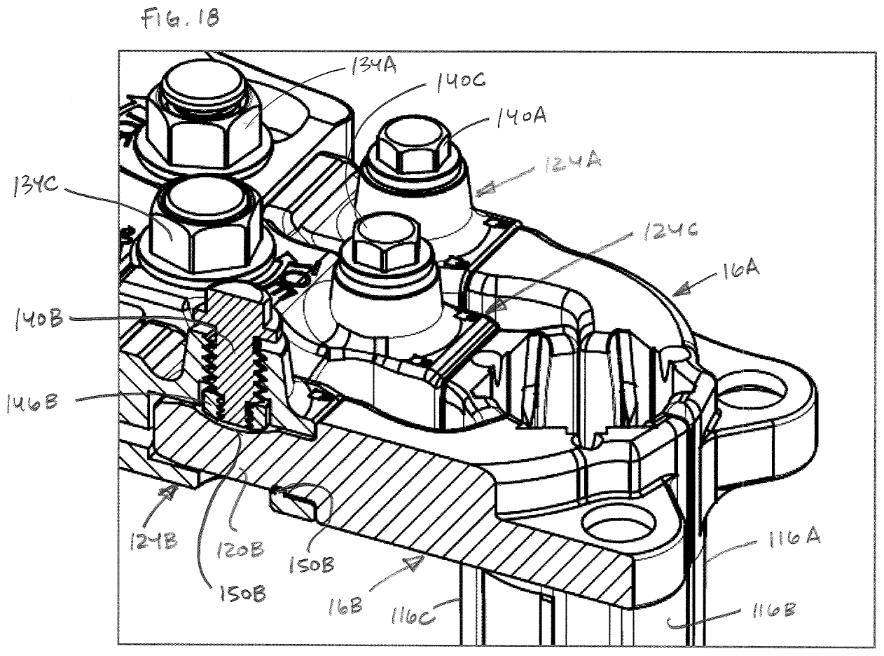

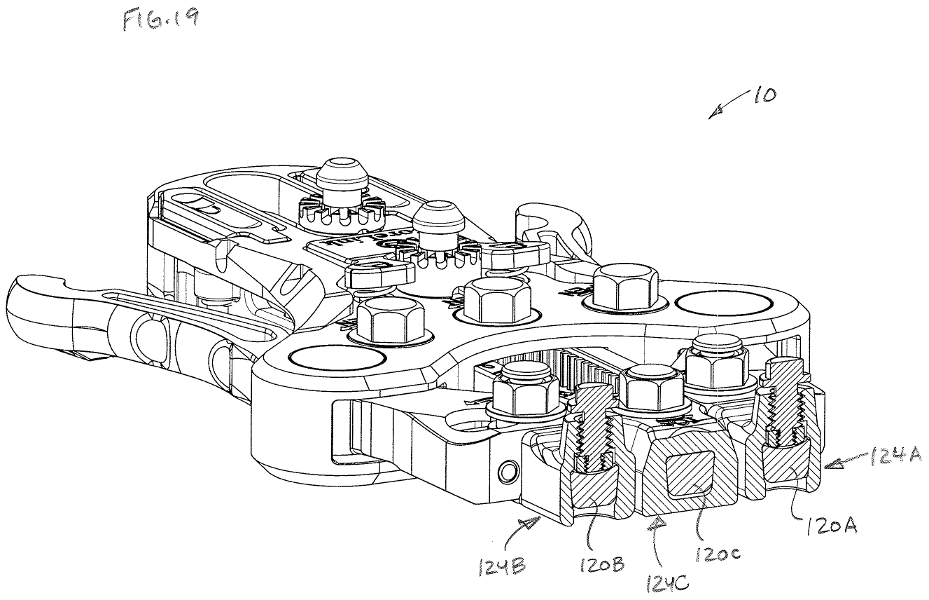

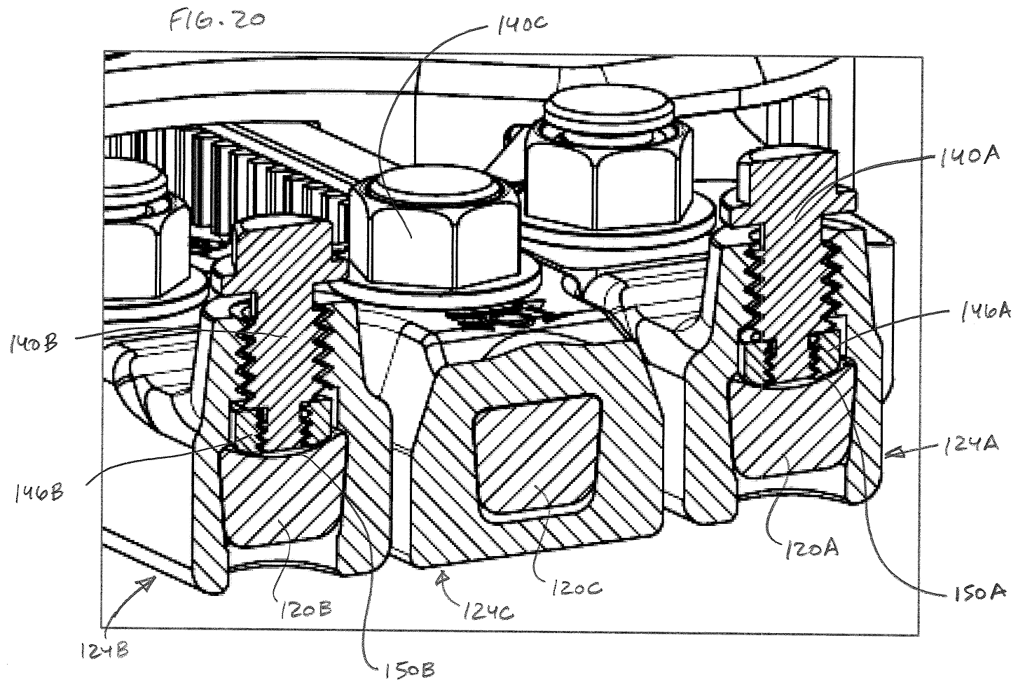

Referring to FIGS. 19 and 20, the illustrated tongue 120A, 120B, 120C of each blade 16A, 16B, 16C tapers from an upper surface toward a lower surface thereof such that the width of the tongue at an upper portion is greater than a width of the tongue at a lower portion, giving the tongue a generally trapezoidal cross-sectional shape. The lug connector 124A, 124B, 124C of each arm 16A, 16B, 16C has a body defining a socket (i.e., a female coupling) having a cross-sectional shape corresponding generally with the cross-sectional shape of the tongue 120A, 120B, 120C and configured to receive the tongue therein, as explained in more detail below. A set screw 140A, 140B, 140C threadably coupled to the lug connector body engages the tongue 120A, 120B, 120C within the socket to secure the tongue within the socket and inhibit the tongue from unintentionally withdrawing from and moving within the socket. The set screw 140A, 140B, 140C may include a hexagonal head or other head for coupling to a tool, such as a wrench or other driver. The set screw 140A, 140B, 140C includes a knob 146A, 146B, 146C (or a convex tip) at the end of its shaft that is sized and shaped to be received in a recess or pocket 150A, 150B (pocket of the middle tongue is hidden) defined by an upper surface of the tongue 120A, 120B, 120C. In addition, as shown in FIG. 18, a locking rib 152B (the other locking ribs are hidden) within each socket is receivable within a groove defined by and extending cross-wise of the tongue 120A, 120B, 120C to further inhibit the tongue 120A, 120B, 120C from unintentionally withdrawing from and moving within the socket.

The cross-sectional size of the socket is such that the tongue 120A, 120B, 120C is insertable into the socket in an upper position relative to the socket so that the tongue clears the locking rib 152B as it is inserted into the socket. With the tongue 120A, 120B, 120C fully inserted in the socket, the set screw 140A, 140B, 140C is tightened to force the tongue downward into a lower position within the socket. The cross-sectional dimension of the socket narrows at the lower portion of the socket to create friction fit between the tongue 120A, 120B, 120C and the socket. The set screw 140A, 140B, 140C may be tightened until the tongue 120A, 120B, 120C bottoms out at the bottom wall of the socket and the rib 152B (other the other ribs not shown) is received in the groove. The combination of the set screw 140A, 140B, 140C, the pocket 150B, the friction fit, and the rib-in-groove 150B inhibits movement of the blade relative to the arm, thereby reducing wobbling of the blade relative to the arm.

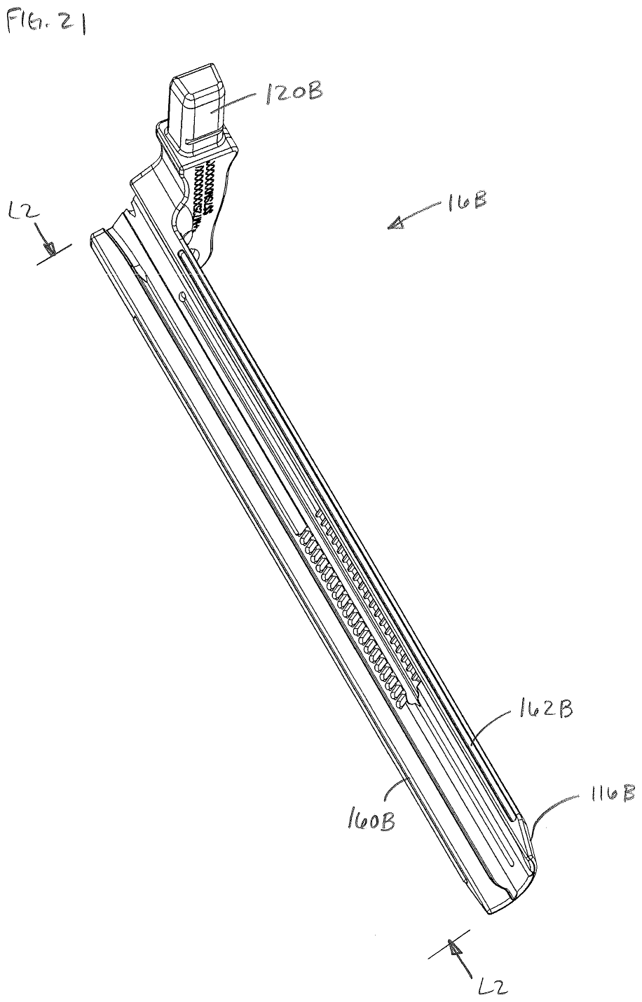

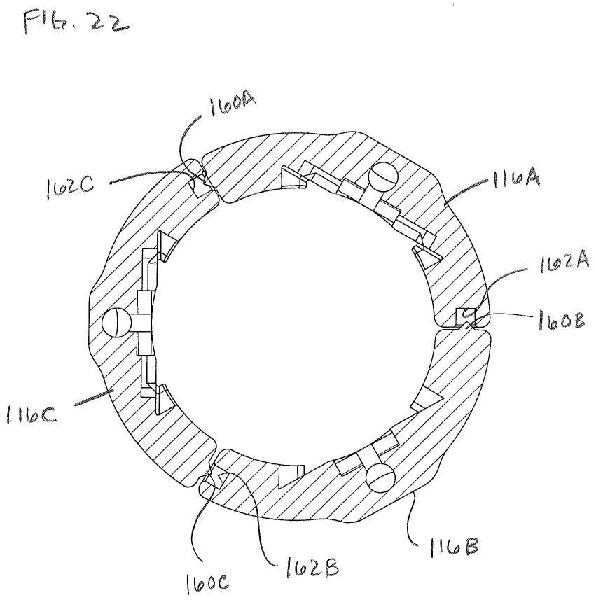

Referring to FIGS. 21 and 22, each blade body 116A, 116B, 116C includes a longitudinal rib 160A, 160B, 160C extending longitudinally on one arcuate side thereof, and a groove 162A, 162B, 162C extending longitudinally on the other, opposite arcuate side. The longitudinal rib 160A, 160B, 160C is configured to mate with a corresponding groove 162A, 162B, 162C of one adjacent blade body 116A, 116B, 116C, and the groove is configured to mate with a corresponding longitudinal rib of the other adjacent blade body. When the blades 16A, 16B, 16C are in the fully closed configuration, the blade bodies 116A, 116B, 116C are mated together to define a substantially closed circumference, as shown in FIG. 22 for example. In the illustrated embodiment, the longitudinal rib 160A, 160B, 160C extends continuously, as a single rib, along at least a majority of the length L2 of the corresponding arcuate side of the blade body 116A, 116B, 116C. The groove 162A, 162B, 162C also extends continuously, as a single groove, along at least a majority of the length of the corresponding arcuate side of the blade body 116A, 116B, 116C. In other embodiments, the groove 116A, 116B, 116C and/or the longitudinal rib 160A, 160B, 160C may not be continuous. Each of the groove 116A, 116B, 116C and longitudinal rib 160A, 160B, 160C may extend at least 50%, or at least about 60%, or at least about 70%, or at least about 75%, or at least about 80%, or at least about 90%, or at least about 95% of the length L2 of the blade body 116A, 116B, 116C.

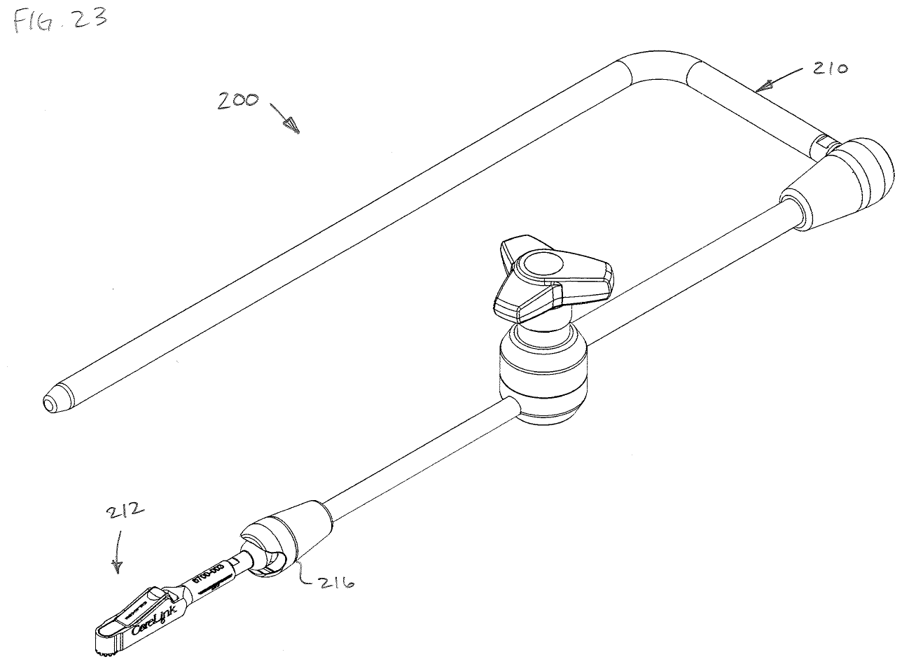

Referring to FIG. 23, an arm mount for the retractor 10, or another retractor, is indicated generally at reference numeral 200. The arm mount 200 is configured to mount the retractor 10 on an operating table or bed or the like to free the hands of the user and assistants. The arm mount includes an arm, generally indicated at 210, and a retractor adaptor, generally indicated at 212, coupled to the arm. The arm 210 may be a conventional arm as is generally known in the art or another type of arm. A coupler 216 of the arm 210 couples the retractor adaptor 212 to the arm and allows articulation of the retractor adaptor relative to the arm, and therefore, articulation of the retractor 10 relative to the arm.

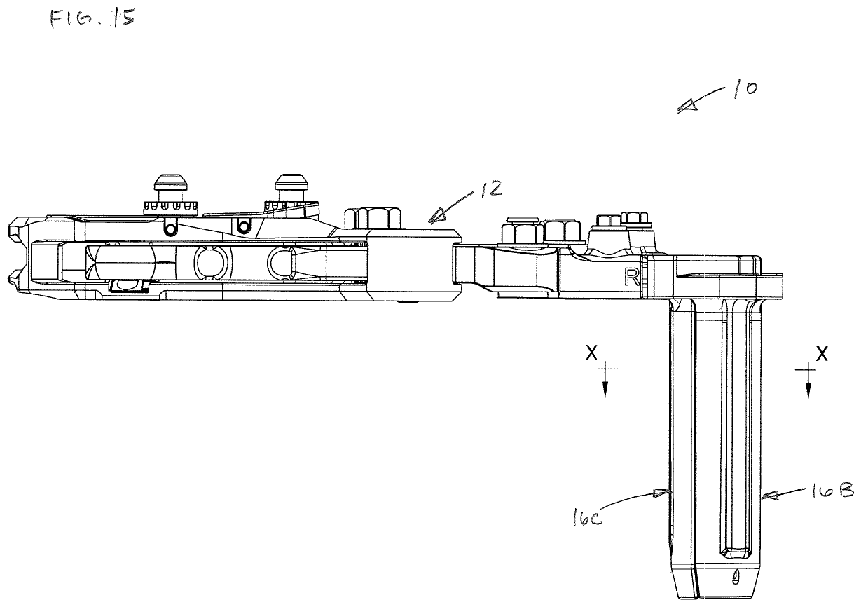

The retractor adaptor 212 is couplable to the retractor 10 via an adaptor mount. The illustrated retractor 10 includes two adaptors mounts: a proximal adaptor mount 220A and a distal adaptor mount 220B, each indicated generally. The proximal adaptor mount 220A is coupled to and moves with the central arm 16C, and the distal adaptor mounts 220B is coupled to the body 12 and does not move with the central arm. Other than these differences, the adaptor mounts 220A, 220B are generally identical. As seen best in FIGS. 14 and 15, each adaptor mounts 220A, 220B includes a stud 224A, 224B including an enlarged button head, and annular row of teeth 226A, 226B surrounding the stud.

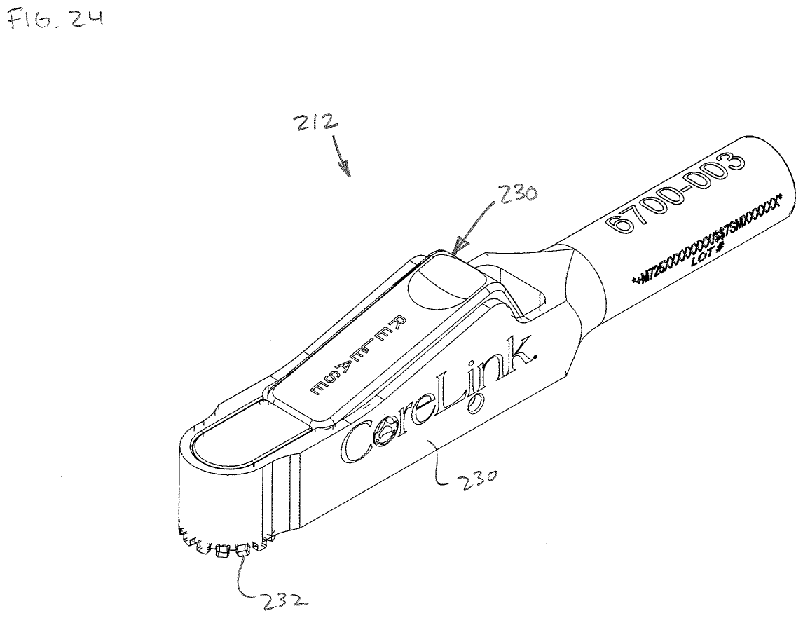

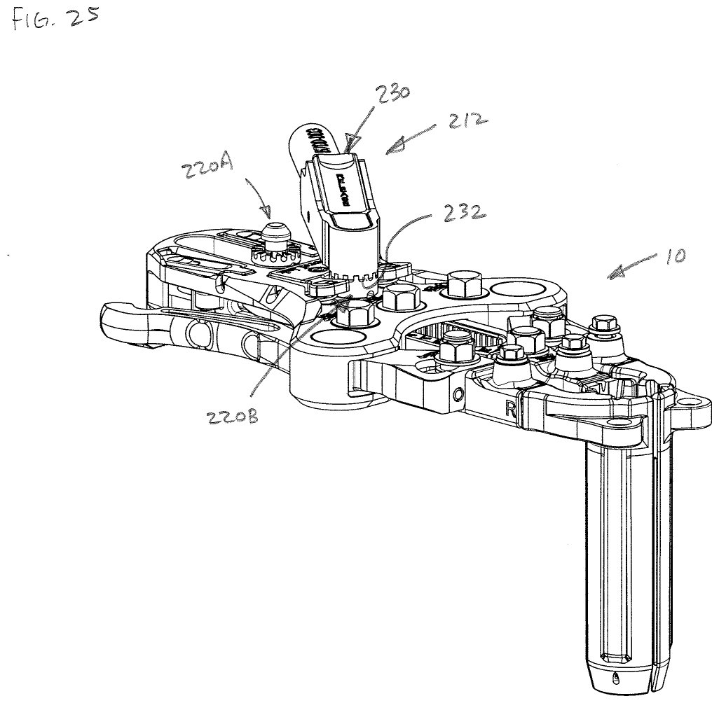

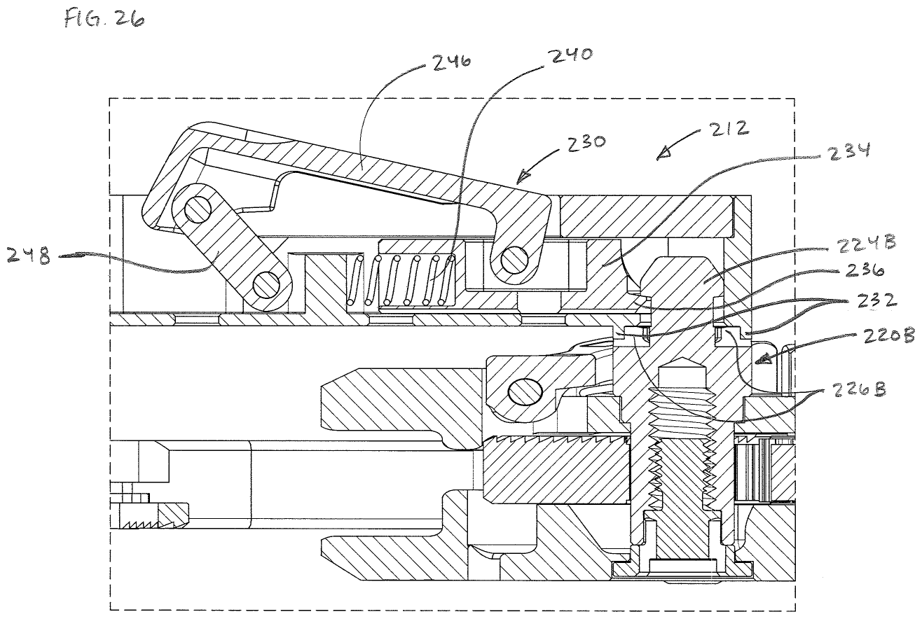

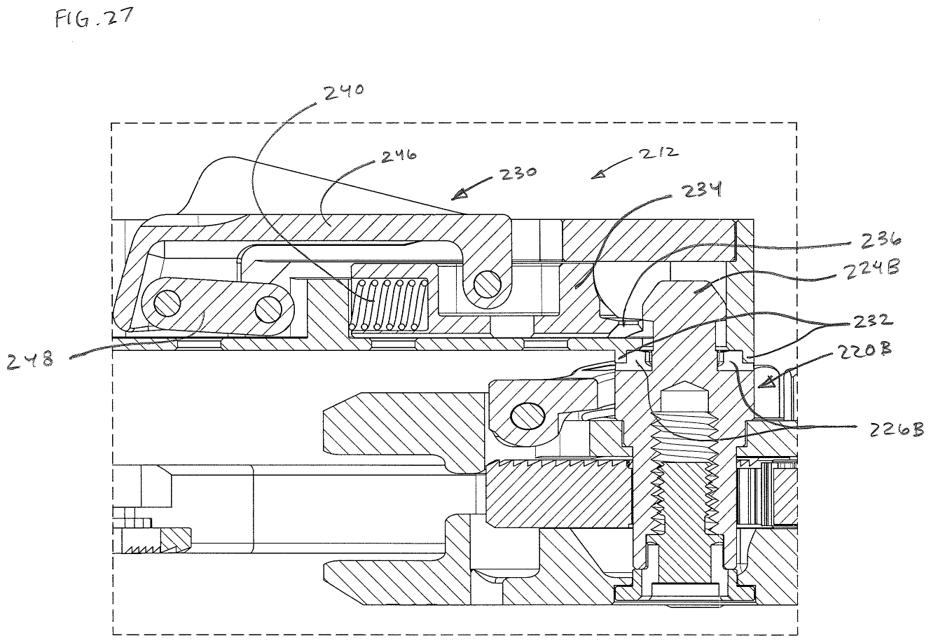

Referring to FIGS. 24 and 25, the retractor adaptor 212 includes an elongate adaptor body 230 having proximal and distal ends. The proximal end of the adaptor body 230 is internally threaded to threadably mate with the coupler 216. The distal end of the body 230 has a lower end including an annular row of teeth 232 configured to mesh and engage the annular row of teeth 226A, 226B of the adaptor mounts 220A, 220B. Referring to FIGS. 26 and 27, the retractor adaptor 212 further includes a latch, generally indicated at 230 configured to releasably latch the adaptor on the stud 224A, 224B. The latch 230 includes a retractable plunger 234 having an arcuate claw 236. The plunger 234 is selectively movable between a latched position, in which the arcuate claw 236 is moved under the button head of the button stud 224A, 224B and inhibits the adaptor 212 from being removed from the stud, and an unlatched position, in which the arcuate claw is disengaged from the button head and enables the adaptor to be removed from the stud.

Referring still to FIGS. 26 and 27, the latch 230 further includes a spring 240 biasing the plunger 234 in the latched position, and a release mechanism configured to enable the plunger to be moved to the unlatched position against the bias of the spring 240. The release mechanism includes a button 246 having one end hingedly coupled to the plunger 234 and an opposite end hingedly coupled to one end of a connecting link 248. The other end of the connecting link 248 is hingedly coupled to the adaptor body 230. In operation, the button 246 is pushed downward so that it rotates about its hinged connection to the plunger, thereby causing the plunger to slide and retract proximally relative to the adaptor body 230. In generally, the release mechanism 242 operates as a slider-crank mechanism, whereby the button 246 functions as the connecting rod, the connecting link functions as the crank (although the crank does not actuate the slider-crank), and the plunger functions as the slider.

In use, the retractor adaptor 212 is positioned over one of the adaptor mounts 220A, 220B in a selected angular position relative to the retractor 10. The adaptor 212 is then latched onto the selected adaptor mount 220A, 220B in the selected angular position, such that the annular row of teeth 232 of the adaptor 212 mesh with the annular row of teeth 226A, 226B of the adaptor mount to inhibit rotation of the adaptor on the adaptor mount. The retractor adaptor 212 is readily removable from the retractor 10 by depressing the button 246 and pulling the adaptor off of the adaptor mount 220A, 220B.

Modifications and variations of the disclosed embodiments are possible without departing from the scope of the invention defined in the appended claims.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A retractor for spine surgery comprising:
a body;
left, right, and center retracting arms coupled to the body; and
left, right, and center blades operatively coupled to proximal ends of the respective retracting arms.

2. The retractor set forth in claim 1, wherein each left and right retracting arms are rotatably drivable independently relative to the body via a corresponding rack and pinion mechanism and corresponding a lever mechanism.

3. The retractor set forth in claim 2, wherein each of the lever mechanisms includes an effort arm and a load arm, wherein the effort arms are configured to nest within the body.

4. The retractor set forth in claim 3, wherein each effort arm includes a ratchet rack engaging a pawl coupled to the body.

5. The retractor set forth in claim 2, wherein the center retracting arm is coupled to a rack and pinion mechanism to enable linear movement of the center retracting arm relative to the body.

6. The retractor set forth in claim 5, wherein each of the rack and pinion mechanisms includes a tool coupler at an upper surface of the body, wherein the tool couplers are configured to couple to a tool to actuate rotation of the corresponding pinion.

7. A retractor adaptor configured to couple to a tissue retractor for spine surgery, the retractor adaptor comprising:
an adaptor body having proximal and distal ends, the proximal end of the adaptor body configured to couple to an arm mount;
an annular row of teeth at the distal end of the adaptor body configured to mesh with an annular row of teeth of an adaptor mount of the tissue retractor; and
a latch configured to releasably latch the retractor adaptor on the adaptor mount.
